(19) United States
(12) Patent Application Publication
(10) Pub. No.: US 2003/0138880 A1
(43) Pub. Date: Jul. 24, 2003

(54) Solid-phase synthesis of oligosaccharides and glycopeptides using glycosynthases

(75) Inventors: Stephen G. Withers, Vancouver (CA); Knud J. Jensen, Copenhagen (DK); Lars Petersen, Copenhagen (DK); Jakob L. Tolborg, Ballerup (DK)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of British Columbia, Vancouver (CA)

(21) Appl. No.: 10/228,629

(22) Filed: Aug. 26, 2002

Related U.S. Application Data
(60) Provisional application No. 60/314,921, filed on Aug. 24, 2001.

(51) Int. Cl. C12P 21/06; C12P 19/28; C12P 19/04

(52) U.S. Cl. 435/68.1; 435/85; 435/101

(57) ABSTRACT

The present invention provides materials and methods for the solid-phase synthesis of oligosaccharides and glycopeptides. Such materials and methods include mutant glycosidase enzymes, or “glycosynthases,” chemically-derivatized acceptor molecules, and specific solid support matrices.

### BACKGROUND OF THE INVENTION

Oligosaccharides play many diverse roles in biology. For example, oligosaccharide modification of proteins and lipids may be required for proper structure and function of these molecules. Extracellularly, oligosaccharides are involved in biological recognition processes such as immune response and infection. A better understanding of such recognition events will assist in the design of new drug candidates against a wide range of illnesses, including cancer and AIDS.

A problem with studying carbohydrate interactions is the limited access to well-defined oligosaccharides. Purification of oligosaccharides from natural sources is tedious, and it is difficult to obtain preparations free of contaminants. It is possible to synthesize oligosaccharides, but no general glycosylation protocol has yet been developed; this makes each individual structure a challenging synthetic target. Accordingly, there is a need for parallel and combinatorial synthesis of oligosaccharides for the study of carbohydrate interactions.

Enzymatic approaches for the preparation of oligosaccharides are interesting alternatives to traditional chemical synthesis, the main advantage being the regio- and stereoselectivity offered by the enzymes without the use of protecting groups. Glycosyltransferases are one type of enzyme used in such enzymatic synthesis methods. Glycosyltransferases are enzymes that transfer a glycosyl moiety from a sugar donor to an acceptor. Acceptor molecules used by glycosyltransferases are typically other sugar moieties, and are chemically attached to the donor molecule through an alcohol functional group. Glycosyltransferases can also use lipids or nucleotides as acceptor molecules. However, nucleotide sugar substrates often used by glycosyltransferases are expensive and the number of glycosyltransferases readily available is not sufficiently substantial.

Glycosidases are glycosyltransferases that use water as an acceptor molecule, and as such, are typically glycoside-hydrolytic enzymes. Glycosidases can be used for the formation of glycosidic bonds in vitro by controlling the thermodynamics or kinetics of the reaction mixture. Even with modified reaction conditions, though, glycosidase reactions can be difficult to work with, and glycosidases tend to give low synthetic yields as a result of the reversible transglycosylase reaction and the competing hydrolytic reaction.

A glycosidase can function by retaining the stereochemistry at the bond being broken during hydrolysis or by inverting the stereochemistry at the bond being broken during hydrolysis, classifying the glycosidase as either a “retaining” glycosidase or an “inverting” glycosidase, respectively. Retaining glycosidases have two critical carboxylic acid moieties present in the active site, with one carboxylate acting as an acid/base catalyst and the other as a nucleophile, whereas with the inverting glycosidases, one carboxylic acid functions as an acid and the other functions as a base.

A more detailed understanding and availability of glycosidases and glycosyltransferases has been rather limited until recently, but is increasing by way of recombinant DNA technology. Solution-phase enzymatic glycosylations using glycosyl transferases have gained increasing attention. These same enzymes have been used for solid-phase oligosaccharide synthesis. Schuster et al., J. Am. Chem. Soc. 116: 1135-1136 (1994); Halcomb et al., J. Am. Chem. Soc. 116: 11315-11322 (1994); Blixt et al., J. Carbohydr. Chem. 16: 143-154 (1997); Blixt et al., J. Org. Chem. 63: 2705-2710 (1998); Blixt et al., Carbohydr. Res. 319: 80-91 (1999).

A glycosynthase is a mutated retaining glycosidase, wherein typically, one of the active site amino acid carboxylate-side chain nucleophiles is replaced by a non-nucleophilic amino acid side chain; Mackenzie et al. J. Am. Chem. Soc. 120: 5583-5584 (1998). The glycosidase thus loses its ability to hydrolyze glycosidic bonds, but it can instead catalyse the glycosylation of sugar acceptors using glycosyl fluoride donors (FIG. 1). Various additional glycosynthases have now been reported. Fort, et al. J. Am. Chem. Soc. 122: 5429-5437 (2000); Malet, et al. FEBS Lett. 440: 208-212 (1998); Mayer, et al., FEBS Lett 466 40-44 (2000), Trincone, et al., Bioorg. Med. Chem. Lett. 10: 365-368 (2000); Nashiru, et al., Angew. Chem. Int. Ed. 40, 417-420 (2001); Mayer, et al., Chemistry and Biology 8: 437-443 (2001). Glycosynthases are also described in U.S. Pat. No. 5,716,812 which is incorporated herein by reference.

Glycosynthases show great promise for synthesizing therapeutically-useful oligosaccharides with highly-specific stereochemistry and regiochemistry, and with specifically desired oligomeric size and identity. Accordingly, it is desirable to be able to use glycosynthases to synthesize such oligosaccharides in an inexpensive, high-capacity manner. The present invention provides methods and compositions to accomplish this goal.

### BRIEF SUMMARY OF THE INVENTION

The present invention includes the use of nucleic acids, bacterial strains, and methods for the solid-phase enzymatic glycosylation of sugar acceptors. A key feature of the invention therefore is to express a glycosynthase that act to glycosylate a selected sugar acceptor. Glycosynthases of the invention may be created from glycosidases derived from multiple sources, including fungal and bacterial sources.

The invention provides compositions and methods that facilitate a coordinated in vitro glycosyl transfer with a specific donor-acceptor pair. The invention also provides solid supports and methods by which saccharides, polypeptides, lipids, and antibiotics may be attached to such solid supports. Further, the invention provides methods for the synthesis of oligosaccharides, glycopeptides, and glycolipids.

One embodiment of the invention provides a method for solid-phase synthesis of an oligosaccharide or a glycopeptide, including the step of enzymatically glycosylating a saccharide or polypeptide acceptor molecule attached to a solid support, with the enzymatic glycosylation catalyzed by a glycosynthase enzyme. In another embodiment of the invention, a saccharide attached to a solid support is a monosaccharide. In still another embodiment of the present invention, the saccharide attached to a solid support is an oligosaccharide of at least two monosaccharide units. In yet another embodiment of the invention, the acceptor molecule attached to a solid support is an amino acid. And in still another embodiment of the present invention, the acceptor molecule attached to a solid support is a polypeptide at least two amino acids in length.

In an aspect of the present invention, an antibiotic, lipid, or polypeptide acceptor molecule is attached to a solid support and enzymatic glycosylation of the acceptor is catalyzed by a glycosynthase enzyme.

One embodiment of the present invention provides a method for solid-phase synthesis of an oligosaccharide, including the step of enzymatically glycosylating an aminophenyl derivative of a saccharide acceptor molecule attached to a solid support, with the enzymatic glycosylation catalyzed by a glycosynthase enzyme. In another embodiment of the invention, an aminophenyl derivative of a saccharide acceptor molecule attached to a solid support is a para-aminophenyl derivative of a saccharide.

In an embodiment of the present invention, the glycosynthase is a mutant glycosidase from Agrobacteriaum sp. In another embodiment of the invention, the Agrobacterium sp. glycosynthase is either a E358S or E358G mutants of Agrobacterium sp. β-glucosidase.

In an embodiment of the invention, the glycosynthase is a mutant glycosidase from Cellulomonas fimi. In another embodiment of the invention, the Cellulomonas fimi glycosynthase is a E519S, E519G or E519A mannosidase 2A mutant.

In an embodiment of the invention, the glycosynthase is a mutant glycosidase from Humicola insolens. In another embodiment of the invention, the Humicola insolens glycosynthase is a E198A, E198S or E198G Ce17B mutant.

In an embodiment of the invention, the glycosynthase is a mutant glycosidase from Sulfolobus solfataricus. In another embodiment of the invention, the Sulfolobus solfataricus glycosynthase is a E387G, E387A or E387S beta glycosidase.

In an embodiment of the invention, the glycosynthase is a mutant glycosidase from Bacillus licheniformis. In another aspect of the invention, the glycosynthase from Bacillus licheniformis is a E134A, E134S or E134G 1,3-1,4-glucanase mutant.

In one aspect of the present invention, a solid support used in a glycosynthase-based synthetic method of the invention is a polyethylene glycol polyacrylamide copolymer. In another aspect of the invention, an acceptor molecule is chemically attached to the polyethylene glycol polyacrylamide copolymer solid support used in a glycosynthase-based synthetic method of the invention.

### BRIEF DESCRIPTION OF THE DRAWINGS

For purpose of illustrating the invention, there are depicted in the drawings certain embodiments of the invention. However, the invention is not limited to the precise arrangements and instrumentalities of the embodiments depicted in the drawings.

FIG. 1 is a scheme depicting the generalized glycosylation mechanism of glycosynthases with α-D-galactosyl fluoride as the donor and X representing the side chain of the mutated residue.

FIG. 2 is a reaction scheme for solid phase attachment of aminophenyl glucosides and subsequent glycosylation using a glycosynthase.

FIG. 3 is a reaction scheme for glycosylation of an exemplary PEGA-resin bond pentapeptide.

### DETAILED DESCRIPTION OF THE INVENTION

The present invention provides a method for solid-phase synthesis of an oligosaccharide or a glycopeptide. In accordance with the method of the invention, a saccharide or peptide acceptor molecule may be selectively derivatized and attached to a solid support that does not interfere with desired subsequent reactions. The immobilized acceptor molecule can then be enzymatically glycosylated using a glycosynthase enzyme. A glycosynthase enzyme useful in the present invention may be isolated from natural sources. Alternatively, the glycosynthase enzyme of the invention may be a glycosidase that contains at least one altered amino acid, resulting in the enzyme's inability to catalyze glycosyl hydrolysis while retaining the ability to catalyze glycosyl transfer with a suitable donor-acceptor pair.

Definitions

The articles “a” and “an” are used herein to refer to one or to more than one (i.e. to at least one) of the grammatical object of the article. By way of example, “an element” means one element or more than one element.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.” Conventional notation is used herein to describe polynucleotide sequences: the left-hand end of a single-stranded polynucleotide sequence is the 5′ end; the left-hand direction of a double-stranded polynucleotide sequence is referred to as the 5′-direction.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. As used in the present invention, the term “polypeptide” can refer to a sequence of as little as two amino acids linked by a peptide bond, or an unlimited number of amino acids linked by peptide bonds.

The term “protein” typically refers to large polypeptides.

The term “peptide” typically refers to short polypeptides.

A “mutant” polypeptide as used in the present application is one which has the identity of at least one amino acid altered when compared with the amino acid sequence of the naturally-occurring protein. Further, a mutant polypeptide may have at least one amino acid residue added or deleted to the amino acid sequence of the naturally-occurring protein.

Conventional notation is used herein to portray polypeptide sequences: the left-hand end of a polypeptide sequence is the amino-terminus; the right-hand end of a polypeptide sequence is the carboxyl-terminus.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The term “saccharide” refers in general to any carbohydrate, i.e., a chemical entity having the most basic structure of (CH₂O)ₙ. Saccharides vary in complexity, and may also include nucleic acid, amino acid, or virtually any other chemical moiety existing in biological systems.

“Monosaccharide” refers to a single unit of carbohydrate of a defined identity.

“Oligosaccharide” refers to a molecule consisting of several units of carbohydrates of defined identity. Typically, saccharide sequences between 2-20 units may be referred to as oligosaccharides.

“Polysaccharide” refers to a molecule consisting of many units of carbohydrates of defined identity. However, any saccharide of two or more units may correctly be considered a polysaccharide.

A “glycosyl transferase” is an enzyme that transfers a glycosyl moiety from a sugar donor to an acceptor. Acceptor molecules used by glycosyltransferases are typically other sugar moieties, and are attached through an alcohol functional group, but glycosyltransferases can also use lipids or nucleotides as acceptor molecules.

A “glycosidase” is a glycosyltransferase that uses water as an acceptor molecule, i.e., a glycoside-hydrolytic enzyme. Glycosidases can also be used for the formation of glycosidic bonds in vitro by controlling the thermodynamics or kinetics of the reaction mixture. A glycosidase that retains the stereochemistry at the hydrolyzed bond is referred to as a “retaining” glycosidase and a glycosidase that inverts the stereochemistry at the hydrolyzed bond is referred to as an “inverting” glycosidase.

A “glycosynthase” is a mutated retaining glycosidase with one of the active site carboxylate nucleophiles typically replaced by a non-nucleophilic amino acid side chain. A glycosynthase lacks the ability to hydrolyze glycosidic bonds, but it can instead catalyze the glycosylation of sugar acceptors using an appropriate donor molecule, such as glycosyl fluoride.

A “solid support” used for a glycosynthase reaction of the present invention is typically a high molecular weight molecule that is inert with respect to the glycosynthase reaction. The solid support has appropriate functional groups that allow chemical coupling of acceptor groups for the glycosynthase reaction.

A “donor” molecule as used herein is a molecule that is chemically coupled to another molecule by the action of an enzyme such as a glycosyltransferase or a glycosynthase.

An “acceptor” molecule as used herein is a molecule that is chemically bonded to a donor molecule by the enzymatic action of a glycosyltransferase or a glycosynthase. An acceptor molecule as used herein is preferably chemically coupled to a solid support.

Unless otherwise specified, the term “derivative” as used herein typically refers to a chemical compound containing an additional chemical moiety. For example, toluene is a methyl derivative of benzene.

SEQ ID NO: 1 illustrates full-length Agrobacterium faecalis beta-glucosidase cDNA, and the corresponding protein is set forth in SEQ ID NO: 2. SEQ ID NO: 3 illustrates the cDNA for the E358S mutant of the A. faecalis beta-glucosidase, and the corresponding mutant protein, a glycosynthase, is set forth in SEQ ID NO: 4.

The methods of the invention should not be construed to be limited solely to a nucleic acid comprising SEQ ID NO: 3, but rather, should be construed to encompass any nucleic acid encoding the polypeptide of SEQ ID NO: 4 or a fragment thereof, either known or unknown, which is capable of catalyzing glycosyl transfer to an appropriate acceptor molecule. Modified nucleic acid sequences, i.e. nucleic acid sequences having sequences that differ from the nucleic acid sequences encoding the naturally-occurring proteins, are also encompassed by methods and compositions of the invention, so long as the modified nucleic acid still encodes a protein having the biological activity of catalyzing glycosyl transfer to an appropriate acceptor molecule, for example. These modified nucleic acid sequences include modifications caused by point mutations, modifications due to the degeneracy of the genetic code or naturally occurring allelic variants, and further modifications that have been introduced by genetic engineering, i.e., by the hand of man. Thus, the term nucleic acid also specifically includes nucleic acids composed of bases other than the five biologically occurring bases (adenine, guanine, thymine, cytosine and uracil).

Nucleic acids having at least 90 percent identity to SEQ ID NO: 3 are also encompassed by the methods and compositions of the present invention. The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator address http://www.ncbi.nlm.nih.gov/BLAST/. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.ov.

In another aspect, a nucleic acid useful in the methods of the present invention and encoding a glycosynthase polypeptide may have at least one nucleotide inserted into the naturally-occurring nucleic acid sequence. Alternatively, an additional glycosynthase protein may have at least one nucleotide deleted from the naturally-occurring nucleic acid sequence. Further, a glycosynthase nucleic acid useful in the invention may have both a nucleotide insertion and a nucleotide deletion present in a single nucleic acid sequence encoding the enzyme.

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. As is known to one of skill in the art, nucleic acid insertions and/or deletions may be designed into the gene for numerous reasons, including, but not limited to modification of nucleic acid stability, modification of nucleic acid expression levels, modification of expressed polypeptide stability or half-life, modification of expressed polypeptide activity, modification of expressed polypeptide properties and characteristics, and changes in glycosylation pattern. All such modifications to the nucleotide sequences encoding such proteins are encompassed by the present invention.

It is not intended that methods of the present invention be limited by the nature of the nucleic acid employed. The target nucleic acid encompassed by methods and compositions of the invention may be native or synthesized nucleic acid. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89.

Fragments of nucleic acids encoding smaller than full-length protein are also included in the present invention, provided the protein expressed by the nucleic acid retains the biological activity of the full-length protein.

The “biological activity of a glycosynthase” is the ability to transfer a glycosyl moiety from a donor moiety to an appropriate acceptor molecule. The acceptor molecule in the present invention may be a saccharide, a nucleotide, a lipid, or a polypeptide.

Therefore, a nucleic acid encoding a smaller than full-length glycosynthase is included in the present invention provided that the “smaller than full-length” glycosynthase has glycosynthase biological activity. Fragments of glycosynthase nucleic acids of the present invention can range from 300 bp to 3600 bp.

In another aspect, the invention may include an isolated nucleic acid of the present invention cloned into a DNA vector. In one aspect of the invention, A. faecalis glycosynthase DNA is cloned into a pET-29b(+) expression vector to create a construct useful in the present invention. Other vectors may be useful in the present invention, including, but not limited to pTUG and pTZ. In another aspect of the invention, A. faecalis glycosynthase DNA is cloned into an expression vector downstream of the 3′ end of a sequence encoding a functional tag. The 5′-end fusion to the A. faecalis glycosynthase comprises a six-histidine sequence to aid in purification of the expressed polypeptide.

In yet another aspect of the present invention, A. faecalis glycosynthase DNA is expressed in E. coli BL21(DE3) cells, using an appropriate expression vector. However, as evidenced by the literature relevant to the art, one skilled in the art will appreciate that A. faecalis glycosynthase DNA can also be expressed in other bacterial cells, including, but not limited to W2244, JM101, and TB-1. A. faecalis glycosynthase protein encoded by nucleic acids useful in the present invention may be expressed using any technique well-known in the art, such as simple expression, high level expression, or overexpression (Sambrook et al., 1989, Molecular Cloning: A Laboratory Manual, Cold Spring Harbor Laboratory, New York). Selection and use of promoters useful in the expression of a glycosynthase of the present invention include lacZ and T7, but one of skill in the art would undoubtedly know how to select and use other such promoters.

The nucleic acids useful in methods and compositions of the invention may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

SEQ ID NO: 4 illustrates full-length A. faecalis glycosynthase polypeptide useful in methods and compositions of the present invention.

The present invention also includes an isolated polypeptide comprising an A. faecalis glycosynthase molecule. Preferably, the isolated polypeptide comprising an A. faecalis glycosynthase molecule is at least about 90% homologous to a polypeptide having the amino acid sequence of SEQ ID NO: 4, or some fragment thereof. More preferably, the isolated polypeptide is about 95% homologous, and even more preferably, about 99% homologous to SEQ ID NO: 4, or some fragment thereof. Most preferably, the isolated polypeptide comprising a A. faecalis glycosynthase molecule is SEQ ID NO: 4.

The present invention also provides for analogs of proteins or peptides which comprise A. faecalis glycosynthase as disclosed herein. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

As used in the specification and claims hereof, the term “glycosynthases” refers to mutant forms of retaining glycosidases with one of the active site carboxylate nucleophiles replaced by a non-nucleophilic amino acid side chain. As a result of this mutation; the glycosidase loses its ability to hydrolyze glycosidic bonds, but it can instead catalyse the glycosylation of sugar acceptors using glycosyl fluoride donors. Such mutations are typically referenced based on the start codon encoding the first (“number one”) amino acid of the polypeptide. Specific examples of glycosynthases which may be used in the present invention include, but are not limited to E358S and E358G mutants of Agrobacterium sp. β-glucosidase (referred to herein as “Abg E358S” and “Abg E358G”, respectively), Cellulomonas fimi Mannosidase 2A mutant E519S, E519G and E519A; Humicola insolens, Cel7B E198A, E198S and E198G; Sulfolobus solfataricus Beta glycosidase E387G, E387A and E387S and Bacillus licheniformis 1,3-1,4-glucanase E134A, E134S and E134G. Glycosynthases may also be utilized which are derived from other retaining enzymes, for example other β-glucosidases, β-galactosidases, β-mannosidases, β-N-acetyl glucosaminidases, β-N-acetyl galactosaminidases, β-xylosidases, β-fucosidases, cellulases, xylanases, galactanases, mannanases, hemicellulases, amylases, glucoamylases, β-glucosidases, β-galactosidases, β-mannosidases, β-N-acetyl glucosaminidases, β-N-acetyl galactosaminidases, β-xylosidases, β-fucosidases, and neuraminidases/sialidases such as those from:

Agrobacterium sp., Bacillus sp., Caldocellum sp., Clostridium sp., Escherichia coli, Kluveromyces sp., Klebsiella sp., Lactobacillus sp., Aspergillus sp., Staphylococcus sp., Lactobacillus sp., Butyrovibrio sp., Ruminococcus sp., Sulfolobus sp., Schizophyllum sp., Trichoderma sp., Cellulomonas sp., Erwinia sp., Humicola sp., Pseudomonas sp., Thermoascus sp., Phaseolus sp., Persea sp., Fibrobacter sp., Phanaerochaete sp., Microbispora sp., Saccharomyces sp.; Hordeum vulgare, Glycine max. Saccharomycopsis sp., Rhizopus sp., Nicotiana, Phaseolus sp., rat, mouse, rabbit, cow, pig, and human sources.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Fragments of A. faecalis glycosynthase polypeptides are also included in the present invention, provided the protein possesses the biological activity of the full-length protein. The biological activity of A. faecalis glycosynthase is the ability to transfer a glycosyl moiety from a donor moiety to an appropriate acceptor molecule. The acceptor molecule in the present invention may be a saccharide, a nucleotide, a lipid, or a polypeptide.

Therefore, an A. faecalis glycosynthase polypeptide smaller than full-length A. faecalis glycosynthase is included in the present invention provided that the “smaller than full-length” A. faecalis glycosynthase has glycosynthase biological activity.

In another aspect of the present invention, methods comprising an isolated A. faecalis glycosynthase enzyme may include highly purified A. faecalis glycosynthase enzymes. Alternatively, methods comprising the A. faecalis glycosynthase enzymes may include cell lysates prepared from the cells used to express the particular glycosynthase enzymes. Further, A. faecalis glycosynthase enzymes of the present invention may be expressed in one of any number of cells suitable for expression of polypeptides, such cells being well-known to one of skill in the art. Such cells include, but are not limited to bacteria, yeast, insect, and mammalian cells.

Substantially pure protein isolated and obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The method of the invention creates an oligosaccharide or a glycopeptide by addition of a glycosyl group derived from a glycosyl donor species. The glycosyl donor species is selected to arrive at the desired final product. Specific examples of suitable glycosyl donor species include but are not limited to donor glycosyl fluorides, although other groups which are reasonably small and which function as relatively good leaving groups can also be used in place of fluoride. Examples of other glycosyl donor molecules include glycosyl chlorides, acetates, propionates, and pivaloates, and glycosyl molecules modified with substituted phenols. The donor molecules may be monosaccharides, or may themselves contain multiple sugar moieties.

A glycosyl fluoride can be prepared from the free sugar by first acetylating the sugar and then treating it with HF/pyridine. This will generate the thermodynamically most stable anomer of the protected (acetylated) glycosyl fluoride. If the less stable anomer is desired, it may be prepared by converting the peracetylated sugar with HBr/HOAc or with HCl to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g., NaOMe/MeOH). In addition, glycosyl donor molecules, including many glycosyl fluorides can be purchased commercially. Thus a wide range of donor molecules are available for use in the methods of the present invention. Specific examples of such donor molecules include but are not limited to α-D-galactosyl fluoride, β-D-galactosyl fluoride, D- or L-glucosyl fluorides, D- or L-mannosyl fluorides, D- or L-fucosyl fluorides and D- or L-sialyl fluorides. The stereochemistry of the donor and acceptor are suitably selected in combination with the enzyme to provide maximum efficiency. Beta glycosidases require alpha glycosyl donors and prefer beta-linked acceptors. Alpha glycosidases would require beta glycosyl donors and prefer alpha-linked acceptors.

As used in the specification and claims of this application, the term “saccharide” refers to both mono- and oligosaccharides which may be immobilized on the solid support and glycosylated in accordance with the method of the invention to produce longer oligosaccharides. While other attachment methods known in the art may be employed, one embodiment of the invention provides that a saccharide may be attached to the solid support by using the saccharide in the form of an aminophenyl glucoside derivative, and using the Backbone Amide Linker (BAL) methodology for attachment of the aminophenyl glucoside derivative to the solid support. The BAL methodology has been described previously, Jensen et al., J. Am. Chem. Soc. 120: 5441-5452 (1998); Alsina et al., Chem. Eur. J. 5: 2787-2795 (1999) and has been used for solid-phase oligosaccharide synthesis. Tolborg et al., Chem. Commun. 147-148 (2000). Further, aminophenyl derivatives of peptides are also useful in the present invention, and the preparation and use of such derivatives is known to one of skill in the art.

The solid support which is used in the method of the present invention is suitably one which swells extensively in aqueous buffer so that the enzyme has access to the immobilized saccharide or peptide. The support should also be itself unreactive with the enzyme and with the glycosyl donor molecule used. One type of solid support which meets these criteria is polyethylene glycol polyacrylamide copolymers (PEGA) available from Polymer Laboratories (Amherst, Mass.) and described in Meldal, M. Tetrahedron Lett. 33 : 3077-3080 (1992), and Meldal et al., J. Am. Chem. Soc. Chem. Commun. 1849-1850 (1994). The solid support might also be MPEG (polyethylene glycol omega-monomethylether), which is described by Thiem et al., Chem. Commun. 1919-1920 (2000); activated Sepharose B4 as described in Barker et al., Biochemistry 19: 589 (1980) or Norberg et al., J. Org. Chem. 63 2705 (1999), or aminopropyl silica as described in Wong et al., J. Am. Chem. Soc. 116: 11315 (1994).

It has been observed that the buffer concentration employed in combination with solid supports of PEGA resin can have a substantial impact on the yields of glycosylated products. While not intending to be bound by any specific mechanism, it is believed that this results from change in the extent of swelling, leading to differential access of the enzyme to the immobilized saccharide or peptide. In addition, increases in donor concentration to high levels were actually observed to cause a decrease in yield. This is also believed to be due to a swelling effect, with increased donor concentration causing a contraction of the resin, and thus less enzyme access. While not intending to be bound by any specific mechanism, if this understanding is correct, it would be generally desirable to select the combination of support and resin such that the support is expanded or swelled to at least about 90% of its maximum size to provide good permeability for macromolecules of molecular weight comparable to the glycosynthase being used (generally, 60-70 kDa).

In one non-limiting embodiment of the present invention outlined in FIG. 2, using the BAL methodology, amine acceptor derivatives are chemoselectively attached through the 2-amino group to resin bound o-PALdehyde (2) by a reductive amination to form an acid stable secondary amine. After N-acylation to increase the acid-lability of the handle, the amido sugar is efficiently releasable from the safety catch linker with TFA-H₂0 (19:1). Tolberg et al., supra This method is highly effective for anchoring amines in general, however, not all amino saccharide derivatives are equally useful for the subsequent glycosylation step. For example, a glucosamine derivative anchored through the 2-amino group by a BAL handle to low-loading PEGA resin proved to be a poor substrate, presumably due to its bulkiness. Also, cellobiosamine attached to a squarate linker (Tietze et al., Chem. Ber. 124: 1215-1221(1991) as applied by Blixt et al. was a poor substrate.

In an embodiment of the present invention designed to achieve adequate yields of glycosylation products, the present invention makes use of aminophenyl saccharide derivatives. In a non-limiting example, as shown in FIG. 2, o-PALdehyde (2) was used as a handle for the attachment of three regioisomers of aminophenyl β-D-glucoside (3 a-c). The aminophenyl glucoside was reacted with the handle in the presence of NaBH₃CN in AcOH—MeOH (1:99). Glycosylation was achieved through the addition of α-D-galactosyl fluoride (20 mM) and 1.0 mg/ml Agrobacterium glycosynthase in 100 mM NaPi buffer. The product was acylated using Ac₂O-pyridine (2:1) and then released using a standard protocol (TFA-H₂O, 19:1) to yield acetamidophenyl 2,3,4,6-tetra-O-acetyl-β-D-glucosides.

The aminophenyl saccharide acceptor derivatives can be released without prior acetylation, however the acetylation step may be desirable for several reasons. Firstly, the rate of cleavage was lower for the non-acetylated than for the acetylated system. For the acetylated system, a smaller amount (10% of total) of product was observed in the second cycle, whereas for the non-acetylated system, a significant amount (28% of total) was observed. Long cleavage times seemed to yield small amounts of byproducts, and a significant amount of a non-polar compound was formed in the non-acetylated system. Secondly, because of the polar and basic nature of the free amine, an eluent system containing 0.1% TFA was required to obtain integratable peaks. However, the free amine eluted in the injection peak, thus hampering accurate integration at 215 nm. More accurate results were obtained at 268 mm, a local 1ₘₐₓ for 4-aminophenyl-β-D-glucopyranoside, but the absorbance at this wavelength was much lower, thus demanding injection of larger amounts of compound. Thirdly, the stability of this free amine was also questionable as seen in the workup problems and seemingly low yield for the synthesis.

As discussed below in the examples, higher yields of oligosaccharide product were obtained when the aminophenyl glucoside used was the para regioisomer. Thus, in a preferred embodiment of the invention, the aminophenyl glucoside acceptor used is the para regioisomer.

The specific saccharide which is employed will depend on the desired product. Specific, non-limiting examples of saccharides which may be employed in the invention include the aminophenyl derivatives of galactose, mannose, sialic acid, N-acetyl glucosamine and glucosamine. In an embodiment of the invention, a synthetic method may be performed in multiple cycles to add several glycosyl groups. If the same glycosyl group is to be added, the ratio of donor to bound acceptor would be increased to provide material for multiple cycles. In another embodiment of the invention where different glycosyl moieties are to be added, the donor species could be protected, for example with acetyl, benzoyl, alloyl or benzyl protective groups. Each cycle would then include the steps of removal of excess donor, deprotection and addition on the next donor species.

For purposes of producing a glycopeptide, in one embodiment of the invention, a synthetic method starts with a peptide or a glycopeptide attached to a solid support. A peptide may be attached to a support such as PEGA using the BAL methodology. A suitable peptide will generally have a length of from 2 to 200 amino acids, for example, 2 to 5 amino acids. The peptide may be assembled from Fmoc-protected amino acid pentafluorophenyl esters as described in Jansson et al., J. Chem. Soc. Perkin Trans. 1: 1001-1006 (1996) and standard peptide coupling reagents. Glycosylation can be arranged to occur on Asn, Gln, Hyl, Tyr, Ser, Thr or Cys residues, or on saccharides attached to them. The position of glycosylation can be controlled through the use of glycosynthases specific of particular amino acids or through the use of specific protecting groups.

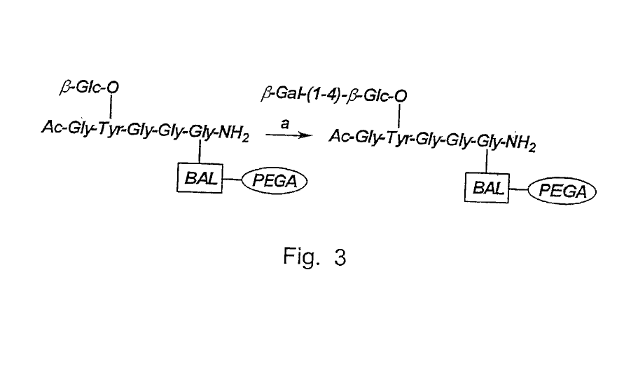

FIG. 3 is a reaction scheme for glycosylation of an exemplary PEGA-resin bound pentapeptide. In this exemplary reaction, the PEGA-resin bound pentapeptide is treated with a glycosynthase (for example 1.0 mg/ml Abg E358G) and a glycosyl donor (for example 20 mM α-D-galactosyl fluoride) in an aqueous buffer (for example 100 mM pH 9 0 NaPi buffer). The resulting glycopeptide can be removed by dilute acid, for example TFA:H₂0 (19:1), with or without prior protection be peracetylation.

### EXPERIMENTAL DETAILS

The invention is now described with reference to the following examples. These examples are provided for the purpose of illustration only and the invention should in no way be construed as being limited to these examples but rather should be construed to encompass any and all variations which become evident as a result of the teaching provided herein.

PEGA Solid Support Resin

Low-loading (LL, 0.2 mmol/g) PEGA resin, which has excellent swelling abilities in water (31.7 mL/g), and a good permeability towards macromolecules (60-70 kDa), was used in the experiments described below unless otherwise indicated. High-loading (HL, 0.4 mmol/g) PEGA resin was also tested in some cases: The swelling (15.5 mL/g) and permeability are poorer because of shorter PEG chains. In addition, tests were conducted on cross-linked ethoxylate actylate resin (CLEAR) from Peptides International, which is a hydrophilic, macroporous resin with a high degree of cross-linking resulting in a poorer swelling (5.5 mL/g) and permeability. As indicated below, all but low-loading PEGA proved inefficient, presumably because of low accessibility by the glycosynthase.

If PEGA resin is dried after a normal washing procedure, it becomes very sticky and difficult to handle. For this reason, PEGA was purchased swelled in methanol and the manufacturer recommends handling the resin only swelled in an appropriate solvent depending on the reaction in which it is to be used. However, this was not well suited for glycosylation reactions where small amounts of resin typically are weighed off numerous times. If the swelled resin either loses solvent due to evaporation or takes up water from the air caused by hygroscopic resin or solvent, the effective loading will vary to an unsatisfactory degree. Therefore it was desirable to develop a way of handling the resin in the dry state. Polyethylene glycol (PEG) is insoluble in diethyl ether, a fact that is exploited in the use of MPEG soluble supports, and it was reasoned that the PEGA resin could be crystallized or collapsed in a controlled way by the treatment with ether. After a normal washing procedure, successively increasing concentrations of diethyl ether in DCM (1×5 mm with 25%, 50%, and 75% solutions) caused the resin to collapse gradually. Finally, it was washed with neat diethyl ether (2×5 min each) and dried in vacuo to constant weight. This resin was slightly sticky, but firm, bead shaped, and easier to handle than PEGA in the swelled state. Small quantities (10-20 mg) were readily handled with plastic spatulas. Glass or metal should be avoided. In the following, all loadings are based on resins treated in the above-described way.

### EXAMPLE 1

### Synthesis of Aminophenyl Glucosides

4-Aminophenyl-β-D-glucopyranoside 3a:

4-Nitrophenyl 2,3,4,6-tetra-O-acetyl-b-D-glucopyranoside (3.75 g, 8 mmol) was deprotected by Zemplén deacetylation (MeOH, 150 mL, and cat. NaOMe) over night. The solution was neutralized with AcOH to pH 5, Pd/C added (ca. 280 mg, 10% Pd based on dry weight, contains 50% water) and hydrogenated over night at 1 atm. Upon filtration through Celite and concentration, the crude syrup turned brown on standing. The coloration was removed by flash chromatography on silica gel, but returned upon evaporation of appropriate fractions. The syrup was crystallized from EtOH and a brownish solid was collected by filtration (0.87 g, 40%). Pure by TLC (Rf 0.55; DCM-acetone-MeOH 6:2:2) and gave a bright pink color with ninhydrin.

An aliquot was treated with Ac₂O-pyridine (3:1) and characterized as the per-acetate: Colorless foam; no clear mp. (in correlation with lit.); ¹H-NMR (400 MHz, CDCl1₃) d 7.67 (1H, s), 7.28 (2H, ddd, J=2.1, 3.2, 9.0 Hz), 6.90 (2H, J=2.1, 3.2, 9.0 Hz), 525 (IH, t, J=9.4 Hz), 5.20 (1H, dd, J=7.5, 9.4 Hz), 5.12 (1H, dd, J=9.2, 9.8 Hz), 4.97 (1H, d, J=7.5 Hz), 4.25 (1H, dd, J=5.3, 12.3 Hz), 4.11 (1H, dd, J=2.3, 12.3 Hz), 3.80 (1H, ddd, J=2.3, 5.2, 9.9 Hz), 2.10 (3H, s), 2.03 (3H, s), 2.02 (3H, s), 2.00 (3H, s), 1.98 (3H, s); ¹³H-NMR (100 MHz, CDCl₃) d 170.5, 170.1, 169.4, 169.3, 168.4, 153.3, 133.6, 121.5, 117.6, 99.5, 72.6, 71.9, 71.1, 68.2, 61.8, 24.2, 20.6 (two C), 20.5 (two C); Anal. calcd. for C₂₂H₂₇NO₁₁: C 54.88, H 5:65, N 2.91; found C 55.06, H 5.69, N 3.05; MS: m/z calcd. for C₂₂H₂₈NO₁₁: 482.17 (M+H), found 482.2; UV: 245 nm (ε17.6×10³ M⁻¹cm⁻¹).

3-Aminopheny1-β-D-glucopyranoside 3b:

2,3,4,6-Tetra-O-acetyl-a-D-glucosyl bromide (1.23 g, 3.00 mmol) in acetonitrile (5 mL) was added to a suspension of 3-nitrophenol (0.501 g, 3.60 mmol), silver carbonate (0.496 g, 1.8 mmol), and DIEA (0.627 mL, 3.60 mmol) in acetonitrile (10 mL). After reaction over night under Ar, the reaction mixture was concentrated. The residue was dissolved in EtOAc (100 mL), extracted with 1M H₂SO₄ (2×25 mL), 0.5M NaOH (2×15 mL), and brine (2×25 mL), dried (MgSO₄), and concentrated. One recrystallization from EtOH-H₂O (2:1) yielded 0.846 g (60%) of a colorless solid (98% pure by RP-HPLC, 215 nm). This material was deprotected and hydrogenated as described for 3a, which upon concentration yielded an oil that crystallized from EtOH and was lyophilized (petroleum ether) to yield an essential quantitative yield containing a small amount of sodium acetate. Pure by TLC (R_{f}0.57; DCM-acetone-MeOH 6:2:2) and gave a bright pink color with ninhydrin.

An aliquot was treated with AC₂O-pyridine (3:1) and characterized as the per-acetate: Colorless foam; no clear mp.; ¹H-NMR (400 MHz, CDCl₃) d 7.40 (2H, m); 7.18 (1H, t, J=8.1 Hz), 704 (1H, J=7.9 Hz), 6.71 (1H, d, J=7.9 Hz), 5.29-5.20 (2H, m), 5.14 (1H, dd, J=9.2, 9.6 Hz), 5.07 (1H, d, J=7.4 Hz), 4.27 (1H, dd, J=4.7, 12.3 Hz), 4.15 (1H, dd, J=1.5, 12.3 Hz), 3.89-3.80 (1H, m), 2.13 (3H, s), 2.04 (3H, s), 2.03 (3H, s), 2.01 (3H,s), 2.00 (3H, s); ¹³H-NMR (100 MHz, CDCl₃) d 170.6, 170.2, 169.4, 169.3, 168.3, 157.2, 139.2, 129.7, 114.3, 112.7, 108.7, 99.0, 72.8, 72.1, 71.1, 68.2, 61.8, 24.6, 20.6 (four C); Anal. calcd. for C₂₂H₂₇N0₁₁: C 54.88, H 5.65, N 2.91; found C 54.59, H 5.78, N 2.98; MS: m/z calcd. for C₂₂H₂₃N0₁₁: 482.17 (M+H), found 482.2; Uv: 243 nm (ε14.8×10⁻¹ M³cm⁻¹), 278 nm (ε3.42×10³ M⁻¹ cm−1).

2-Aminopheny1-β-D-g1ucopyranoside 3c:

2-Nitrophenyl-β-D-glucopyranoside (Sigma) was hydrogenated as described for 3a. After removal of catalyst and solvent, an essential quantitative yield of a white solid was obtained directly; again contained small amounts of salts. Pure by TLC (R_{f}. 0.45, DCM-acetone-MeOH 6:2:2) and produced a bright pink color upon reaction with ninhydrin.

An aliquot was treated with Ac₂O-pyridine (3:1) and characterized as the per-acetate: Colorless crystals; mp. 181-183° C. (lit._Ref511014855\f\h² 185-187° C.); ¹H-NMR (400 MHz, CDCI₃) d 8 36 (1H, dd, J=1.5, 8.1 Hz), 7.82 (1H, s), 7.04 (1H, dd, J=1.5 Hz, 8.1 Hz), 6.97 (1H, dt, J=1.5 Hz, 8.1 Hz 6.91 (1H, dd, J=1.5, 8.1 Hz), 5.34(1H, dd, J=9.0, 9.8 Hz), 5.28 (1H, dd, J=7.5, 9.8 Hz), 5.12 (1H, dd, J=9.1, 9.3 Hz), 5.00 (1H, d, J=7.6Hz), 4.31 (1H, dd, J=5.4, 12.4Hz), 4.14 (1H, dd, J=2.4, 12.4Hz), 3.88 (1H, ddd, J =2.3, 5.4, 12.4 Hz), 2.18(3H, s), 2.05 (6H, 2xs), 2.03 (6H, s); ¹³H-NMR (100 MHz, cDCl₃) d 170.4, 170.4, 169.9, 169.4, 168.6, 145.0, 128.9, 123.8, 123.4, 120.3, 113.7, 99.8, 72.1, 71.8, 71.4, 68.2, 61.7, 60.3, 24.5, 20.8, 20.6, 20.5 (two C); Anal calcd. for C₂₂H₂₇NO₁₁: C54.88, H 5.65, N 2.91; found C 55.06, H0.87, N 2.87; MS: m/z calcd. for C₂₂H₂₃NO₁₁: 482.17 (M+H), found 482.4; UV: 243 nm (s.13.9×10³ M⁻¹ cm⁻¹, 278 nm (s 3.85×10³ M−1 cm⁻¹).

### EXAMPLE 2

### Preparation of Resins

PEGA resin (loading of 0.20 mmol/g; swollen in MeOH; 9.41% by weight) was washed with 2×DMF, 2×DIEA-DMF (1:49), 1×DMF. o-PALdehyde was coupled using a standard peptide coupling protocol by dissolving o-PALdehyde, BOP, and HOBt (2.0 equiv each) DMF. DIEA(5 equiv) was added, allowed to stand for 5 min and the reaction mixture was added to the above resin. After shaking for 16 hours (400 min⁻¹), the resin was washed with 3×DMF, 3×DCM-MeOH (2:1), and 5×DCM. Kaiser test (ninhydrin) at this point was clearly negative, but an acetylation with Ac₂O-pyridine (2:1) for 16 hours was maintained nevertheless. After another washing (3×DMF, 3×DCM-MeOH (2:1), 5×DCM), one of three regioisomers of aminophenyl-β-D-glucopyranoside prepared in Example 1 (3a-c, 5 equiv) was coupled by reductive amination in the presence of NaBH₃CN (10 equiv) in AcOH—MeOH (1:99). After shaking for 16 hours, resins were washed again (3×DMF, 3×DCM-MeOH (2:1), and 5×DCM) and collapsed with diethyl ether in DCM as in the above mentioned sequences.

### EXAMPLE 3

### Solid Phase Glycosylations

A first set of experiments was performed to determine whether the concentration of buffer would have any effect on the rate of glycosylation. Thus, the concentrations of donor (Ga1F, 20 mM) and enzyme (1.0 mg/mL) were kept constant and the concentration of phosphate buffer was 0, 1 mM, 10 mM, or 100 mM (pH 7.0; 1 and 10 mM made by diluting the 100 mM buffer). Enzyme was prepared using a Pharmacia Hi-Trap desalting column equilibrated in the desired buffer, thus eliminating salts from stock solution. Resin (20 mg) was weighed off in 3 mL disposable syringes fitted with at polypropylene filter and a Teflon stopcock. Ga1F was weighed off in 1.5 mL centrifuge tubes, dissolved in the desired buffer and added the desalted enzyme solution. This reaction mixture was then added to the resin. Reactions were shaken at 400 mm⁻¹ for 24 h and then washed: Nanopure water (5 times), DMF (3 times), DCM-MeOH (3 times), and DCM (5 tims), acetylated (Ac₂O-pyridine, 600 ml and 300 ml) for 16-18 hours, washed again (only organic solvents) and finally cleaved as above. Obtained data are presented in Table 1.

TABLE 1
Solid-phase glycosylations of para, meta, and ortho systems in different
concentrations of phosphate buffer (pH 7.0).

| | NaPi | Wave- | | | |
|---|---|---|---|---|---|
| | conc. | length | Loading (mmol/g) | | |
| Acceptor | (mH) | (nm) | Acceptor (%) | Product (%) | Total (%)^{a,c} |
| Para | 0 | 245 | 34 | 66 | 0.084 (57) |
| | | 280 | 34 | 66 | 0.082 (57) |
| Para | 1 | 245 | 34 | 66 | 0.080 (54) |
| | | 280 | 35 | 65 | 0.078 (54) |
| Para | 10 | 245 | 60 | 40 | 0.117 (80) |
| | | 280 | 61 | 39 | 0.114 (79) |
| Para | 100 | 245 | 81 | 19 | 0.129 (88) |
| | | 280 | 82 | 18 | 0.128 (88) |
| Meta | 0 | 243 | ca. 80^{b} | ca. 20^{b} | 0.010 (8) |
| | | 278 | ca. 80^{b} | ca. 20^{b} | 0.010 (8) |
| Meta | 1 | 243 | ca. 85^{b} | ca. 15^{b} | 0.015 (13) |
| | | 278 | ca. 85^{b} | ca. 15^{b} | 0.015 (13) |
| Meta | 10 | 243 | 00 | 0 | 0.100 (84) |
| | | 278 | 100 | 0 | 0.101 (87) |
| Meta | 100 | 243 | 100 | 0 | 0.103 (87) |
| | | 278 | 100 | 0 | 0.103 (90) |
| Ortho | 0 | 243 | —^{b} | —^{b} | —^{b} |
| | | 278 | ca. 80^{b} | ca. 20^{b} | 0.010 (8) |
| Ortho | 1 | 243 | —^{b} | —^{b} | —^{b} |
| | | 278 | ca. 80^{b} | ca. 20^{b} | 0.011 (9) |
| Ortho | 10 | 243 | 100 | 0 | 0.088 (75) |
| | | 278 | 100 | 0 | 0.086 (72) |
| Ortho | 100 | 243 | 100 | 0 | 0.097 (83) |
| | | 278 | 100 | 0 | 0.098 (82) |

^{a}Calculated from loadings in Table 1.
^{b}Integration uncertain because of low intensity.
^{c}Cleavage times of 30-45 minutes. It was later on discovered that 60 minutes was preferable.

For the ortho and meta systems, no significant conversion was observed in buffer concentrations higher than 10 mM NaPᵢ. For 0 and 1 mM some glycosylation was observed; but unfortunately, total cleaved amounts were only around 10% and thus very unsatisfactory. This was believed to be caused by cleavage by liberated hydrogen fluoride. For the para systems, results were more promising; the highest conversions were observed for 0 or 1 mM of >60%. Unfortunately, the total cleaved amount accounts for only 54-57% for the same reasons as discussed above. The 10 mM and 100 mM experiments established around 40% and 20% conversion, respectively, which was far better than the two other systems (ortho and meta). The total cleaved amounts were almost equal with the expected amounts. These experiments provided two major conclusions: The para system demonstrated higher reactivity than meta and ortho, and secondly, the para series demonstrated that the buffer concentration has a profound effect on the efficiency of glycosylation, i.e., the lower the buffer concentration, the higher the conversion to product. Another conclusion of more analytical character is that both wavelengths were equally good for monitoring reaction progress.

The experiment was repeated in 100 mM NaPi but without prior desalting of the enzyme. The resulting data are presented in Table 2.

TABLE 2
Solid-phase glycosylation without desalting enzyme prior to reaction.
Reaction medium of 100 mM NaPi (pH 7.0).

| | | Wave- | Loading (mmol/g) | | |
|---|---|---|---|---|---|
| | NaPi conc. | length | Acceptor | Product | |
| Acceptor | (nM) | (nm) | (%) | (%) | Total (%)^{a,b} |
| Para | 100 | 245 | 86 | 14 | 0.129 (88) |
| | | 280 | 87 | 13 | 0.124 (86) |
| Meta | 100 | 243 | 100 | 0 | 0.109 (92) |
| | | 278 | 100 | 0 | 0.108 (94) |
| Ortho | 100 | 243 | 100 | 0 | 0.099 (85) |
| | | 278 | 100 | 0 | 0.100 (84) |

^{a}Calculated from loadings in Table 1.
^{b}Cleavage times of 30-45 minutes. It was later on discovered that 60 minutes was preferable.

Again, no significant conversion was observed for the ortho and meta systems and the determined loading yielded almost full recovery for all three systems. In the case of the para system only, a low conversion was observed (13-14%), and this conversion was slightly lower than that obtained for the experiment including prior enzyme desalting. From these experiments, compared with data in Table 2, it can be concluded that desalting the glycosynthase enzyme before use increases the extent of enzymatic conversion. Others have observed 58% conversion under similar conditions.

Glycosylation reactions were also performed using solid CaCO₃ (pH 7.0) in place of the phosphate buffer. The results are summarized in Table 3.

TABLE 3
Solid-phase glycosylation with desalting enzyme prior to reaction.
Reaction buffered by solid CaCO3 (pH ca. 7).

| | | Wave- | Loading (mmol/g) | | |
|---|---|---|---|---|---|
| | CaCO₃ equiv. | length | Acceptor | Product | |
| Acceptor | (to GAl-f) | (nm) | (%) | (%) | Total (%)^{a,b} |
| Para | 5 | 245 | 45 | 55 | 0.129 (88) |
| | | 280 | 45 | 55 | 0.128 (88) |
| Meta | 5 | 243 | 100 | 0 | 0.101 (85) |
| | | 278 | 100 | 0 | 0.101 (88) |
| Ortho | 5 | 243 | 100 | 0 | 0.106 (91) |
| | | 278 | 100 | 0 | 0.108 (91) |

^{a}Calculated from loadings in table 1.
^{b}Cleavage times of 60 minutes.

### EXAMPLE 4

Tests were conducted on glycosylation of resins which had been dried prior to derivatization with the aminophenyl glucoside, and those which were maintained in a swelled state until derivatization was complete. Enzyme (1.0 mg/ml of either Abg E3585 or Abg E358G), 20 mM α-D-galactosyl fluoride in 100 mM NaPᵢ buffer (pH 7.0) were combined with resin dried prior to derivatization, resin maintained in a swelled state, and resin dried after derivatization for a period of 24 hours and then worked up using the procedure of Example 3. Drying the resin prior to derivatization resulted in a substantial reduction in glycosylation rates. On the other hand, as summarized in Table 4, drying after derivatization did not substantially alter the results. In both cases, good yields of glycosylated product were obtained.

TABLE 4
Enzymatic glycosylation of dried vs. non-dried resin.

| Entry | Acceptor | Enzyme | Product^{a} | Recovery^{b} |
|---|---|---|---|---|
| 1 | 4a, non-dried | Abg E358S | 63% | NA^{c} |
| 2 | 4a, non-dried | Abg E358G | 79% | NA^{c} |
| 3 | 4a, dried | Abg E358S | 63% | 91% |
| 4 | 4a, dried | Abg E358G | 80% | 92% |

^{a}Mono-/disaccharide ratio.
^{b}Total amount of mono- and disaccharide compared to determined loading.
^{c}Not available, as weighing off swelled PEGA resin is inaccurate for small amounts.

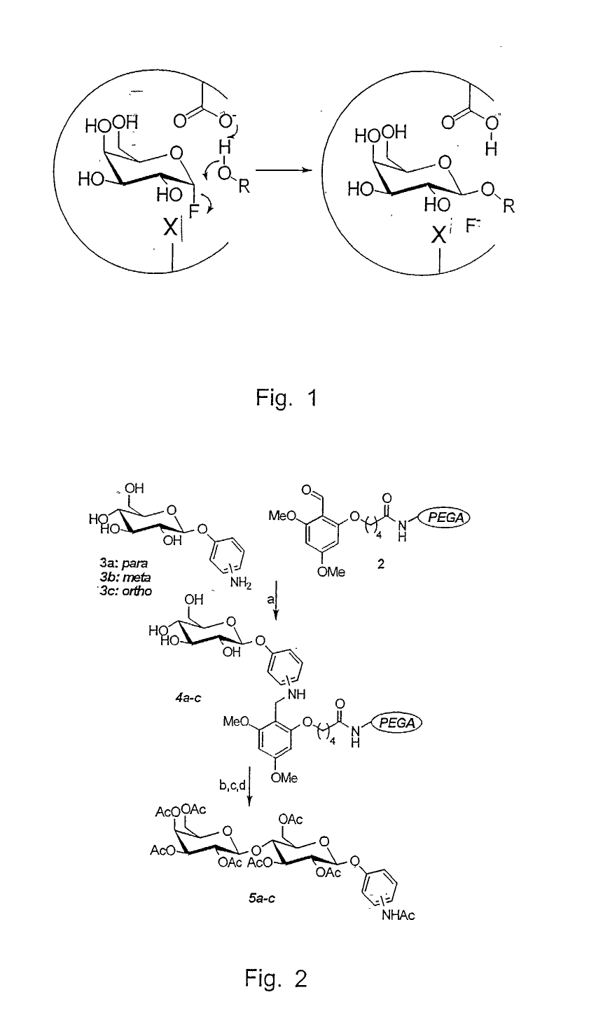

The above results also demonstrated that the glycine mutant, Abg E358G, in each case catalyzed glycosylation at a higher rate than the serine mutant, Abg E3585. This is an interesting observation since it was expected that the serine side-chain via hydrogen bonding could mediate the leaving of fluoride anions from the donor (FIG. 1). In the glycine mutant, water molecules in the free space might perform this more efficiently than can the serine side chain.

### EXAMPLE 5

Having established that Abg E358G was the more efficient of the two glycosynthases tested, further studies revealed that more alkaline buffers were advantageous, since Abg glycosynthases were acid-sensitive but tolerated pH>7 well. At pH 9.0, 100 mM sodium phosphate buffer (NaPᵢ) again proved to be the most useful (Table 5). A high degree of conversion to product was achieved after 24 hours with a full recovery of material, and better than 90% glycosylation was achieved after 72 hours.

TABLE 5
Enzymatic glycosylation of 4a using Abg E358G.

| | | Reaction | | |
|---|---|---|---|---|
| Entry | Acceptor | time | Product^{a} | Recovery^{b} |
| 1 | 4a, non-dried | 24 h | 83% | 98% |
| 2 | 4a, non-dried | 48 h | 90% | 90% |
| 3 | 4a, dried | 72 h | 91% | 90% |

^{a}Mono-/disaccharide ratio.
^{b}Total amount of mono- and disaccharide compared to determined loading.

### EXAMPLE 6

### Synthesis of Glycosylated Aminophenyl Glucoside for Comparison Purposes

4-Acetamidophenyl 2,3,6-tri-O-acetyl-4-(2,3,4,6-tetra-O-acetyl-β-n-galactosyl)-β-D-glucopyranoside 5a:

4-Aminopheny1-β-D-g1ucopyranoside 3a (27.2 mg, 1.0 mmol) and α-D-galactosyl fluoride 1 (21.9 mg, 1.2 mmol) were dissolved in NaPᵢ buffer (100 mM, pH 8.0, 1232 ml), Abg E3585 stock solution (5.6 mg/mL, 268 mL; total 1.5 mg enzyme) was added, and the solution was shaken for 17 h at 400 min¹. TLC (EtOAc-MeOH-H₂O 7:2:1): R_{f} 0.32 (gave a bright pink color with ninhydrin). No trace of starting material could be observed (R_{f} 0.80). Reaction mixture was concentrated to dryness and acetylated with Ac₂O-pyridine (2:1). The reaction mixture was poured over crushed ice and extracted with CH₂Cl₂ (2×50 mL). Combined organic phases were washed with 1M H₂SO₄ (50 mL), aq. NaHCO₃ (50 mL), dried (MgSO₄), and concentrated to a glass (67 mg). The product was separated from a byproduct formed during the prolonged acetylation by flash chromatography on silica gel using EtOAc/petroleum ether (PE). Compound having R_{f} 0.67 (EtOAc) was isolated and crystallized from ether/PE, giving 33.1 mg (43%) of title compound as a colorless solid. No clear mp. (approx. 110-130° C.); ¹H-NMR (500 MHz, CDCl₃) d 7.41 (1H, d, J=9.0 Hz), 7.09 (1H, s; NH), 6.95 (1H, d, J=9.0 Hz), 5.36 (1H, dd, J=0.7, 3.4 Hz; H-4′), 5.28 (1H, t, J=9.0 Hz; H-3), 5.15 (1H, dd, J=8.1, 9.0 Hz; H-2), 5.13 (1H, dd, J=7.7, 10.2 Hz; H-2′), 4.99 (1H, d, J=8.1 Hz; H-1), 4.97 (1H, dd, J=3.4, 10.2 Hz, H-3′), 4.52 (1H, d, J=7.7Hz; H-1′), 4.56 (1H, dd, J=2.1, 11.0Hz; H-6ₐ), 4.17-4.15 (2H, m; H-6_{b}, H-6ₐ′), 4.10 (1H, dd, J=7.7, 11.5 Hz; H-6_{b}′), 3.89 (1H, t, J=9.4 Hz; H-4), 3.88(1H, dd, J=4.0, 7.7 Hz; H-5′), 3.76 (1H, ddd, J=1.5, 2.3, 5.9 Hz; H-5), 2.17, 2.16, 2.09, 2.08, 2.07, 2.07, 2.06, 1:973 (total: 24H, 8xs); ¹³H-NMR (100 MHz, CDCl₃) d 170.4, 170.3, 170.1, 170.0, 169.7, 169.6, 169.1, 168.2, 153.5, 133A, 121.5, 117.7, 101.1, 99.3, 76.2, 72.9, 72:8, 71.5, 71.0, 70.8, 69.1, 66.6, 62.0, 60.8, 24.5, 20.8-20.5 (m); Anal. calcd. for C₃₄H₄₃NO₁₉: C 53.06, H 5.63, N 1.82; found C 52.96, H 5.81, N 1.90; MS: m/z calcd. for C₃₄H₄₃NNaO₁₉: 792.2 (M+Na), found 791.8.

The title compound was identical with product obtained from solid-phase glycosylations with respect to HPLC retention times.

### EXAMPLE 7

An attempt to use only the most accessible sites on the resin (˜30%) was made by downloading, or ‘underloading’, PEGA (0.2 mmol/g) in the coupling step with o-PALdehyde. Thus, only 0.35 equiv. of the handle precursor was used together with 0.7 equiv. BOP and HOBt, respectively, and 4 equiv. DIEA in DMF. This resin, after acetylation and treatment as above, was split into three equal amounts and coupled with the three amines in reductive aminations (5 equiv. amine and. 10 equiv. NaBH₃CN). Aliquots were again acetylated and cleaved, and calculated loadings are listed in Table 6.

TABLE 6
Loadings of downloaded PEGA.

| | Gravimetrical yield | Wavelength | Loading | | |
|---|---|---|---|---|---|
| Acceptor | (%)^{a} | (nm) | mmol/g | %^{b} | % |
| Para | NA | 245 | 0.049 | 74 | 27 |
| | | 280 | 0.047 | 71 | 26 |
| Meta | NA | 243 | 0.050 | 76 | 28 |
| | | 278 | 0.050 | 76 | 28 |
| Ortho | NA | 243 | 0.051 | 77 | 28 |
| | | 278 | 0.051 | 77 | 28 |

^{a}Mass increment after reductive amination. Not available because the actual increase is only a few mg.
^{b}Calculated from a maximum possible acceptor-loading of 0.066 mmol/g (limit from amount of o-PALdehyde).
^{c}Calculated from maximal loading based on an initially 0.2 mmol/g, i..e. ca. 0.18 mmol/g.

As it was the aim of this experiment to obtain a resin with around 30% of maximal loading, this strategy was successful to provide the desired resin-loading. An overall efficiency of 71-77% was observed for the two steps.

The downloaded resins carrying ortho, meta, and para configured acceptors were then subjected to the same glycosylation conditions described above (Table 2) to compare the reactivity of acceptors on fully loaded and downloaded resins, respectively. The results are presented in Table 7. Because monitoring at either wavelength yields the same results, calculations are hereafter stated for the wavelength with highest intensity only (245 nm).

TABLE 7
Solid-phase glycosylation on downloaded PEGA with desalting
enzyme prior to reaction.
Reaction buffered by 100 mM NaPᵢ (pH 7.0) (p. 26)

| | Wavelength | Loading (mmol/g) | | |
|---|---|---|---|---|
| Acceptor | (nm) | Acceptor (%) | Product (%) | Total (%)^{a,b} |
| Para | 245 | 58 | 42 | NA |
| Meta | 245 | 84 | 16 | NA |
| Ortho | 245 | 93 | 7 | NA |

^{a}Total loading not available because of uncertain integration.
^{b}Cleavage times of 60 minutes.

Again, the para configured acceptor yielded the most efficient transglycosylation with 42% conversion as compared with only 16% and 7% for the meta and ortho systems, respectively. However, the effect on downloading was positive as higher conversion was achieved for all three acceptor systems as compared with ˜20% for para and 0% for meta and ortho (Table 1).

### EXAMPLE 8

The positive effect from downloading was then combined with the absence of buffer salts in the reaction medium. Thus, downloaded resins from Table 5 were subjected to transglycosylation in the presence of solid CaCO₃ using Abg B358S and GalF as before. The results are presented in Table 8.

TABLE 8
Solid-phase glycosylation on downloaded PEGA with desalting
enzyme prior to reaction.
Reaction buffered by solid CaCO₃ (pH ca. 7). (p. 27)

| | Wavelength | Loading (mmol/g) | | |
|---|---|---|---|---|
| Acceptor | (nm) | Acceptor (%) | Product (%) | Total (%)^{a,b} |
| Para | 245 | 19 | 81 | NA |
| Meta | 245 | 57 | 43 | NA |
| Ortho | 245 | 81 | 19 | NA |

^{a}Product identity not verified for meta and ortho; minor byproducts were observed in all three.
^{b}Total loading not available because of uncertain integration.
^{c}Cleavage times of 60 mm.

A high conversion was again observed for the para acceptor (˜80%), whereas meta and ortho yielded lower conversions (˜40% and ˜20%, respectively) as compared with para. Thus, it was firmly concluded that the para configured acceptor was by far the most efficient in transglycosylation reactions. For results in both Table 6 and 7, the low intensity of the individual peaks together with poor quality of the analytical column at the time somewhat hampered the exact integration and determination of total cleaved amounts. However, the ratio of acceptor to product was fairly accurate.

### EXAMPLE 9

### Peptide Synthesis

PEGA resin (3.21 g, swelled in MeOH, 9.41% w/w) was washed and derivatized with PALdehyde as described above. Without drying after washing, the resin was subjected to reductive amination with glycine amide hydrochloride (133 mg, 1.20 mmol, 20 equiv) in the presence of NaBH₃CN (78 mg, 1.20 mmol, 20 equiv) in AcOH—MeOH (1 :99). After shaking for 24 h, resin was washed (3×DMF, 3×DCM-MeOH (2: 1), and 5×DCM). The second amino acid was coupled as the symmetrical aithydride: Fmoc-Gly-OH (178 mg, 060 mmol, 10 equiv) and dicyclohexyl carbodiimide(62 mg, 0.30 mmol, 5 equiv) were dissolved in freshly distilled DCM (8 mL). After stirring for 15 minutes, the reaction mixture was filtered and concentrated. Resin was washed with freshly distilled DCM (3×), the symmetrical anhydride in freshly distilled DCM (4 mL) was added, and the mixture shaken for 90 mm followed by washing (3×DCM). The, sequence was repeated once followed by washing (3×DCM and 3×DMF). Remaining amino groups were capped by acetylation (5 mL DMF-Ac₂O-DIEA 18:1:1) for 16 hours, followed by washing (3×DCM and 3×DMF). An aliquot of resin was dried and treated with piperidine-DMF (1:4) for Fmoc-quantification_Ref514061979\f\h¹ (ₘₐₓ 30 l nm, c=7800 cm¹ M-¹) giving a loading of 0.089 mmol/g (50% of max. loading). Remaining resin was deprotected with piperidine-DMF (1:4, 2×5 mm. and 1×15 mm.), then washed (5×DMF). The third amino acid (Gly) was coupled as the pentafluorophenyl (Pfp) ester: Fmoc-Gly-OPfp (85 mg, 0.18 mmol, 3.0 equiv) and HOBt (25 mg, 0.18 mmol, 3.0 equiv) were dissolved in dry DMTF (2 mL) and added to the resin swelled in DMF. After shaking at 400 min⁻¹ for 100 min., the resin was drained, washed twice with DMF and deprotected as above.

The fourth amino acid (glucosylated Tyr) was again coupled as the Pf-ester: Fmoc-Tyr(BzO₄-β-D-Glc)-OPfp (as described in Jensen et al., 1993, J. Chem Soc. Perkins Trans 1: 2119-2129; 134 mg, 0.12 mmol, 2.0 equiv) and HOBt (16 mg, 0.12 mmol, 2.0 equiv) were dissolved in dry DMF (2 mL) and added to the resin. After shaking 400 min⁻¹ for 16 hours, the resin was drained, washed twice with DMF and deprotected as above. The fifth and last amino acid (Gly) was coupled as N-acetylglycine using BOP/HOBt: N-acetylglycine (35 mg, 0.30 mmol, 5.0 equiv), BOP (132 mg, 0.30 mmol, 5.0 equiv), and HOBt (40 mg, 0.30 mmol, 5.0 equiv) was dissolved in dry DMF (2.5 mL). After 5 mm., DIEA was added (103 ml, 0.60 mmol, 10 equiv) and the mixture was added to the resin. After shaking for 16 h, the resin was drained and washed (3×DMF, 3×DCM-MeOH (2:1), and 5×DCM). An aliquot was cleaved (TFA-H₂O 19:1, 1 hour): RP-HPLC (215 nm) indicated purity ˜85% and the identity of the product was established by HR-LSIMS (positive mode): calcd. for C₅₃H₅₃N₆O₁₆ (M+H), 1029.3518, found 1029.3522. Remaining resin was washed (3×MeOH) and deprotected by repeated treatments with NaOMe in MeOH (˜1 mg/ml) until no methyl benzoate could be detected in the liquid. Resin was washed (2×MeOH, 3×DMF, 3×DCM-MeOH (2:1), and 5×DCM). A sample was cleaved and analyzed by HR-LSIMS (positive mode): calcd. for C₂₅H₃₇N₆O₁₂ (M+H), 613.2469, found 613.2470. To improve the chromatographic properties another aliquot of resin was acetylated (Ac₂0-pyridine 2:1, 16 h) and cleaved. RP-HPLC indicated purity ˜94% and the identity of the product was again established by HR-LSIMS (positive mode): calculated for C₃₃H₄₅N₆O₁₆ (M+H), 781.2892, found 781.2885. At this point, remaining resin was collapsed with increasing amounts of diethyl ether in DCM as described above and dried in vacuo.

The remainder of the non-acetylated resin above (199.4 mg) was subjected to enzymatic glycosylation using desalted Abg E358G (1.0 mg/mL) and 20 mg a-D-galactosyl fluoride (36.4 mg, 0.20 mmol) in 100 mM NaPᵢ at pH 9.00 (total 10.0 mL)for 48 hours. After washing, acetylation, and cleavage as above; the residue was treated with dry diethyl ether and concentrated to obtain ˜19 mg of crude product. RP-HPLC indicated 52% conversion and the identity of the product was established by HR-LSIMS (positive mode): calculated for C₄₅H₆₁N₆O₂₄ (M+H), 1069.3737, found to be 1069.3731. Purification of the mixture by preparative HPLC yielded 3.8 mg of monosaccharide glycopeptide and 6.3 mg of disaccharide glycopeptide. This corresponds to yields of 28 and 33%, respectively, or a total of 61% based on Fmoc-quantification of second amino acid.

Each of the two compounds were pre-exchanged in D₂0 (2×30 minutes, followed by concentration to dryness) and finally dissolved in D20 (0.7 mL) for NMR spectroscopy:

Monosaccharide glycopeptide: ¹H-NMR (500 MHz, D₂O) d 7.18 (2H, d, J=8.5 Hz), 6.98 (2H, d, J=8.5 Hz), 5.41 (1H, d, J=8.1 Hz; H-1β), 5.39 (1H, t, J=9.4 Hz; H-3), 5.19 (IH, dd, J=7.7, 8.1 Hz; H-2), 5.14 (1H, t, J=9.4 Hz; H-4), 4.55 (l H, dd, J=6.4, 8.5 Hz; Tyr-Hα), 4.35 (1H, dd, J=4.3, 12.8 Hz; H6ₐ), 4.18 (1H, dd, J=2.1, 12.8 Hz; H-6_{b}), 4.16 (1H, ddd, J=2.1, 4.3, 9.4 Hz; H-5), 3.92-3.73 (8H, m; 8×Gly-Ha), 3.09 (IH, dd, J=6.4, 14.1 Hz; Tyr-Hrβ), 2.94 (1H, dd, J=8.5, 14.1 Hz; Tyr-Hβ), 2.06, 2.05, 2.04, 2.03, 1.94(total 5× acetyl-CH₃); Disaccharide glycopeptide: ¹H-NMR (500 MHz, D₂O, complex spectrum, selected data only) d 7.17 (2H, d, J=8.5 Hz), 6.96 (2H, m), 5.34 (IH, d, J=7.7 Hz; H-1β), 5.27 (IH, t, J=9.0 Hz; H-3), 4.55 (1H, dd, J=6.4, 8.5 Hz; Tyr-Hα), 4.10 (IH, dd, J=9.0, 9.8 Hz; H-4), 3.98 (1H, ddd, J=2.1, 6.0, 9.8 Hz; H-5), 3.93-3.64 (8H, m; Gly-CH₂), 3.09 (1H, dd, J=6.4, 14.1 Hz; Tyr-Hβ), 2.93 (IH, dd, J=8.5, 14.1 Hz; Tyr-Hβ), 2.18-1.93 (24H,m; CH₃).

Based on the above data, it is clear that the glucosyl moiety must be galactosylated on the 4-position. The H—S has a large coupling constant with H-4, and the latter is shifted up-field compared to other signals, which are acetylated. ¹³H-NMR (125 MHz, D₂O, selected data) d 100.9 (β-anomer), 99.1 ((β-anomer, second anomer signal split into two signals due to rotamers).

### EXAMPLE 10

### Design Construction, Expression, and Purification of Glu358Ser Glycosynthase

Plasmid-containing strains were grown in Luria-Bertani broth containing 50 mg/ml kanamycin (LBₖₐₙ) or in TYP (16 g/l tryptone, 16 g/l yeast extract, 5 g/l NaCl, 2.5 g/l K₂HPO₄) containing 50 mg/ml kanamycin (TYPₖₐₙ). Pyrococcus woesei (Pwo) DNA polymerase and deoxynucleoside triphosphates were from Boehringer Mannheim (Indianapolis, Ind.). Restriction endonucleases and T4 DNA ligase were from New England BioLabs (Beverly, Md.). Electrocompetent Escherichia coli Top10 cells and the pZeroBlunt cloning kit were obtained from Invitrogen (Carlsbad, Calif.). The pET-29b(+) expression vector, electrocompetent E. coli BL21 (DE3) cells and His-binding metal chelation resin were obtained from Novagen (Madison, Wis.). Preparation of oligonucleotide primers and DNA sequencing was performed at the Nucleic Acids and Peptide Service (NAPS) Unit, University of British Columbia.

The gene encoding the Agrobacterium sp. glycosidase (abg) was amplified by the polymerase chain reaction (PCR) and subsequently subcloned into the His₆ fusion protein expression vector, pET-29b(+) (Novagen, Madison, Wis.). The PCR mixture contained 10 μM oligonucleotide primers (shown below), 1 mM concentrations of the four deoxynucleoside triphosphates in 100 ml of DNA polymerase buffer, and 25 ng of plasmid pTug10Nabg, carrying the entire abg gene. After heating the mixture to 95° C., the reaction was started by adding 5 U of Pwo DNA polymerase (Boehringer Mannheim, Indianapolis, Ind.). Thirty PCR cycles (45 s at 94° C., 45 s at 56° C and 70 s at 72° C.) were performed in a thermal cycler (GeneAmp PCR System 2400, Perkin Elmer, Norwalk, Conn.). Agarose gel electrophoresis of the PCR product revealed a single DNA fragment of approximately 1380 bp. The primers were as follows: SEQ ID NO: 5, 5′-AA CAT ATG GGA CCG TTA TGG CTA GAC-3′ (forward primer) and SEQ ID NO: 6, 5′-GAG AAC CTC GAG CCC CTT GGC AAC CCC ATG GTT CC-3′ (reverse primer). Underlined are the restriction sites NdeI and XhoI introduced by the two primers. The PCR product was purified and subsequently subcloned into the cloning vector pZero2.0 as well as the expression vector pET-29b(+) using procedures described elsewhere.

A ‘megaprimer’ PCR approach was used to introduce a serine residue at the position of the catalytic nucleophile (E358). The oligonucleotide primers were as follows. Mutagenic reverse primer, SEQ ID NO: 7, 5′-TT GTA GCA GGC GCC GTT GCT GGT GAT GTA GCA CTC CGG-3' (the mutated codon is underlined), forward primer, SEQ ID NO: 8, 5′-GGT CTT CAA GGG CGA ATA TCC-3′, reverse primer, SEQ ID NO: 9, 5′-TCA CCC CTT GGC AAC CCC ATG-3′. In a first PCR, a 300 bp fragment was amplified using the mutagenic and the forward primer. The PCR product was purified and subsequently used in a second PCR as megaprimer. The conditions for the first PCR were as described above, but pTug10NabgE358C was used as the template. The mixture for the second PCR contained 2.5 μg megaprimer, 10 mM reverse primer as well as 25 ng of plasmid pTug10NabgE358C. The following PCR protocol was used: 5 cycles (60 s at 95° C., 180 s at 72° C.), then 25 cycles (45 s at 95° C, 45 s at 58° C and 80 s at 72° C.). The major band of about 600 bp was purified by agarose gel electrophoresis. The purified PCR fragment was ligated into the vector pZero2.0 and subsequently subcloned into pET29abgHis6. The cloned product, called pET29abgE358S, was sequenced to verify the induced mutation. The vector pET29abgE358A was prepared in a similar manner (data not shown).

Recombinant Abg E358S was purified from IPTG-induced cultures of E. coli BL21(DE3) cells, carrying the plasmid pET29abgE358S. The cell suspension was passed twice through a French press at 5° C., centrifuged at 10000×g for 30 min and the soluble cell extract purified by Ni²⁺ chelation chromatography (His-bind resin, Novagen, Madison, Wis.). A 20 L culture of Abg E358S processed in this way yielded 3.5 g of pure mutant enzyme.

Rescue of the glycosidic bond cleaving activity of each nucleophile mutant was performed as is known in the art, using anionic nucleophiles. Concentrations of mutant enzyme solutions were quantified directly by absorbance at 280 nm (₂₈₀^{0.1%}=2.20). Curve-fitting analysis was performed with the computer program GraFit 3.0.

All transglycosylation kinetic studies were performed at 25° C. Assay solutions consisted of α-D-galactopyranosyl fluoride (α-GaIF) and para-nitrophenyl β-D-glucopyranoside (PNPG) in 150 mM NaPi, 150 mM NaCl, 1 mg/ml bovine serum albumin, pH 7. An Orion fluoride electrode (model 96-09BN), interfaced with a Fischer Scientific (Malvern, Pa.) Accumet 925 pH/ion meter, was used to monitor fluoride release following addition of a small aliquot of Abg E358A or E358S. Final concentrations of enzyme were 0.108 mg/ml (2.12 μM) E358S and 1.94 mg/ml (37.9 μM) E358A. All enzymatic rates were corrected for the spontaneous hydrolysis rate of α-GalF. In the first studies, a fixed concentration of α-GalF (57 mM) was used to determine an initial rate profile for PNPG. A maximal rate was observed at 22 mM PNPG followed by substrate inhibition at higher concentrations of PNPG. These data were fit according to Equation 1 using GraFit.

Vₒ=(Vₘₐₓ[S])/(Kₘ+[S]+([S]²/Kᵢ)) Equation 1

Kᵢ represents the inhibition constant for the substrate (in this case PNPG) binding in a non-productive mode. The complementary experiment was carried out in which PNPG was kept at a constant concentration of 22 mM and the concentration of α-GalF was varied. Apparent values of k_{cat}, Kₘ and k_{cat}/Kₘ for GalF and PNPG were also derived using GraFit.

In studies of the dependence of reaction rate upon pH, the following buffers were used: pH 5-6, 100 mM sodium citrate; pH 7-8, 100 mM NaPi; pH 8-9, 100 mM 3-[(1,1-dimethyl-2-hydroxyethyl)amino]-2-hydroxypropanesulfonic acid. Apparent values of k_{cat}/Kₘ for α-GalF were determined from the linear dependence of rates (Vₒ=k_{cat}Eₒ/Kₘ) measured at three low concentrations of α-GalF (5, 10 and 15 mM) at a fixed concentration of PNPG (22 mM).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for solid-phase synthesis of an oligosaccharide or a glycopeptide comprising the step of enzymatically glycosylating a saccharide or polypeptide acceptor molecule attached to a solid support, wherein the enzymatic glycosylation is catalyzed by a glycosynthase enzyme.

2. The method of claim 1, wherein the saccharide is a monosaccharide.

3. The method of claim 1, wherein the saccharide is an oligosaccharide comprising at least two monosaccharide units.

4. The method of claim 1, wherein the acceptor molecule is an amino acid.

5. The method of claim 1, wherein the acceptor molecule is a polypeptide comprising at least two amino acids.

6. The method of claim 1, wherein the acceptor molecule is an aminophenyl derivative of a saccharide.

7. The method of claim 1, wherein the acceptor molecule is a para-aminophenyl derivative of a saccharide.

8. The method of claim 1, wherein the acceptor molecule is chosen from the group consisting of an antibiotic, a lipid, a polypeptide.

9. The method according to claim 1, wherein the glycosynthase enzyme is a mutant form of Agrobacterium sp. β-glucosidase.

10. The method of claim 9, wherein the β-glucosidase contains an Glu358Ser mutation.

11. The method of claim 9, wherein the β-glucosidase contains an Glu358Gly mutation.

12. The method according to claim 1, wherein the glycosynthase enzyme is a mutant form of Cellulomonas fimi mannosidase 2A.

13. The method of claim 12, wherein the mannosidase contains a Glu519Ser mutation.

14. The method of claim 12, wherein the mannosidase contains a Glu519Gly mutation.

15. The method of claim 12, wherein the mannosidase contains a Glu519Ala mutation.

16. The method according to claim 1, wherein the glycosynthase enzyme is a mutant form of Humicola insolens endoglucanase-1.

17. The method of claim 16, wherein the endoglucanase contains a Glu198Ser mutation.

18. The method of claim 16, wherein the endoglucanase contains a Glu198Gly mutation.

19. The method of claim 16, wherein the endoglucanase contains a Glu198Ala mutation.

20. The method according to claim 1, wherein the glycosynthase enzyme is a mutant form of Sulfolobus solfataricus β-glycosidase.

21. The method of claim 20, wherein the β-glycosidase contains a Glu387Ser mutation.

22. The method of claim 20, wherein the β-glycosidase contains a Glu387Gly mutation.

23. The method of claim 20, wherein the β-glycosidase contains a Glu387Ala mutation.

24. The method according to claim 1, wherein the glycosynthase enzyme is a mutant form of Bacillus licheniformis 1,3-1,4-glucanase.

25. The method of claim 24, wherein the 1,3-1,4-glucanase contains a Glu134Ser mutation.

26. The method of claim 24, wherein the 1,3-1,4-glucanase contains a Glu134Gly mutation.

27. The method of claim 24, wherein the 1,3-1,4-glucanase contains a Glu134Ala mutation.

28. The method of claim 1, wherein the solid support is a polyethylene glycol polyacrylamide copolymer.

29. The method according to claim 28, wherein the acceptor molecule is chemically attached to the polyethylene glycol polyacrylamide copolymer solid support.


SEQUENCE LISTING (9 sequences)

SEQ ID NO: 1 - DNA, 1379 bp, Agrobacterium sp.
atgaccgatcccaacacgctcgcagcccgtttccccggcgatttcctgtttggcgtcgcaactgcctcgttccagatcgaggttccaccaaggcggatggccgcaagccctccatctgggatgccttctgcaatatgccgggccatgtcttcgggcgtcacaatggcgatatcgcctgcgatcattacaatcgctgggaggaagacctcgatctcatcaaggagatgggggtggaggcctatcgtttctcgctcgcctggccgcgcatcattcccgatggtttcgggccgatcaacgagaagggtctggatttctacgaccgtctcgttgatggctgcaaggcacgcgggatcaagacctatgcgacgctgtaccattgggatctgccgctgaccctgatgggggatggcggctgggcttcccgctccacggcacatgccttccagcgttacgccaagaccgtcatggcccgcctaggcgaccggctggatgcggttgcgaccttcaacgagccttggtgcgcggtgtggctcagccatctctatggcgtccacgcgccgggcgagcgcaacatggaggcggcccttgccgccatgcaccatatcaacctcgcccatggtttcggcgtggaagcttcccgccatgtcgcgccgaaagtgccggtggggctggtattgaacgcccattccgctattcccgcctccgatggcgaggctgatctgaaggcggccgagcgggccttccagttccacaatggcgcgttctttgacccggtcttcaagggcgaatatcccgccgagatgatggaagcgctgggtgatcgtatgcctgtcgtggaggcggaagacctcggcatcatcagccagaagcttgactggtggggcctgaattattacacgccgatgcgcgtcgccgacgacgcgacaccgggcgtggaattccccgcgactatgcccgcaccggcggtcagcgatgtgaagaccgatatcggctgggaggtttacgctccggcgctgcatacgctggtcgagacgctctacgagcgttacgacctgccggagtgctacatcaccgaaaacggcgcctgctacaatatgggcgtcgaaaacggccaggtcaatgaccagccgcgtctcgattattacgccgaacacctcggcatcgtcgccgatctgatccgtgacggttacccgatgcgcggttatttcgcctggagcctgatggataatttcgaatgggccgagggttaccgcatgcgtttcgggctcgtgcatgtggattatcagacccaggtgcggacggtgaagaatagcggcaagtggtacagcgcgctggcttcgggttttccgaaggggaaccatggggttgccaaggggtga

SEQ ID NO: 2 - protein, 459 aa, Agrobacterium sp.
MTDPNTLAARFPGDFLFGVATASFQIEGSTKADGRKPSIWDAFCNMPGHVFGRHNGDIACDHYNRWEEDLDLIKEMGVEAYRFSLAWPRIIPDGFGPINEKGLDFYDRLVDGCKARGIKTYATLYHWDLPLTLMGDGGWASRSTAHAFQRYAKTVMARLGDRLDAVATFNEPWCAVWLSHLYGVHAPGERNMEAALAAMHHINLAHGFGVEASRHVAPKVPVGLVLNAHSAIPASDGEADLKAAERAFQFHNGAFFDPVFKGEYPAEMMEALGDRMPVVEAEDLGIISQKLDWWGLNYYTPMRVADDATPGVEFPATMPAPAVSDVKTDIGWEVYAPALHTLVETLYERYDLPECYITENGACYNMGVENGQVNDQPRLDYYAEHLGIVADLIRDGYPMRGYFAWSLMDNFEWAEGYRMRFGLVHVDYQTQVRTVKNSGKWYSALASGFPKGNHGVAKG

SEQ ID NO: 3 - DNA, 1380 bp, Agrobacterium sp.
atgaccgatcccaacacgctcgcagcccgtttccccggcgatttcctgtttggcgtcgcaactgcctcgttccagatcgaaggttccaccaaggcggatggccgcaagccctccatctgggatgccttctgcaatatgccgggccatgtcttcgggcgtcacaatggcgatatcgcctgcgatcattacaatcgctgggaggaagacctcgatctcatcaaggagatgggggtggaggcctatcgtttctcgctcgcctggccgcgcatcattcccgatggtttcgggccgatcaacgagaagggtctggatttctacgaccgtctcgttgatggctgcaaggcacgcgggatcaagacctatgcgacgctgtaccattgggatctgccgctgaccctgatgggggatggcggctgggcttcccgctccacggcacatgccttccagcgttacgccaagaccgtcatggcccgcctaggcgaccggctggatgcggttgcgaccttcaacgagccttggtgcgcggtgtggctcagccatctctatggcgtccacgcgccgggcgagcgcaacatggaggcggcccttgccgccatgcaccatatcaacctcgcccatggtttcggcgtggaagcttcccgccatgtcgcgccgaaagtgccggtggggctggtattgaacgcccattccgctattcccgcctccgatggcgaggctgatctgaaggcggccgagcgggccttccagttccacaatggcgcgttctttgacccggtcttcaagggcgaatatcccgccgagatgatggaagcgctgggtgatcgtatgcctgtcgtggaggcggaagacctcggcatcatcagccagaagcttgactggtggggcctgaattattacacgccgatgcgcgtcgccgacgacgcgacaccgggcgtggaattccccgcgactatgcccgcaccggcggtcagcgatgtgaagaccgatatcggctgggaggtttacgctccggcgctgcatacgctggtcgagacgctctacgagcgttacgacctgccggagtgctacatcacctcgaacggcgcctgctacaatatgggcgtcgaaaacggccaggtcaatgaccagccgcgtctcgattattacgccgaacacctcggcatcgtcgccgatctgatccgtgacggttacccgatgcgcggttatttcgcctggagcctgatggataatttcgaatgggccgagggttaccgcatgcgtttcgggctcgtgcatgtggattatcagacccaggtgcggacggtgaagaatagcggcaagtggtacagcgcgctggcttcgggttttccgaaggggaaccatggggttgccaaggggtga

SEQ ID NO: 4 - protein, 459 aa, Agrobacterium sp.
MTDPNTLAARFPGDFLFGVATASFQIEGSTKADGRKPSIWDAFCNMPGHVFGRHNGDIACDHYNRWEEDLDLIKEMGVEAYRFSLAWPRIIPDGFGPINEKGLDFYDRLVDGCKARGIKTYATLYHWDLPLTLMGDGGWASRSTAHAFQRYAKTVMARLGDRLDAVATFNEPWCAVWLSHLYGVHAPGERNMEAALAAMHHINLAHGFGVEASRHVAPKVPVGLVLNAHSAIPASDGEADLKAAERAFQFHNGAFFDPVFKGEYPAEMMEALGDRMPVVEAEDLGIISQKLDWWGLNYYTPMRVADDATPGVEFPATMPAPAVSDVKTDIGWEVYAPALHTLVETLYERYDLPECYITSNGACYNMGVENGQVNDQPRLDYYAEHLGIVADLIRDGYPMRGYFAWSLMDNFEWAEGYRMRFGLVHVDYQTQVRTVKNSGKWYSALASGFPKGNHGVAKG

SEQ ID NO: 5 - DNA, 26 bp, Artificial Sequence
  PCR Primer
aacatatgggaccgttatggctagac

SEQ ID NO: 6 - DNA, 35 bp, Artificial Sequence
  PCR Primer
gagaacctcgagccccttggcaaccccatggttcc

SEQ ID NO: 7 - DNA, 38 bp, Artificial Sequence
  PCR Primer
ttgtagcaggcgccgttgctggtgatgtagcactccgg

SEQ ID NO: 8 - DNA, 21 bp, Artificial Sequence
  PCR Primer
ggtcttcaagggcgaatatcc

SEQ ID NO: 9 - DNA, 21 bp, Artificial Sequence
  PCR Primer
tcaccccttggcaaccccatg